(19) United States
(12) Patent Application Publication
(10) Pub. No.: US 2018/0303520 A1
(43) Pub. Date: Oct. 25, 2018

(54) METHODS, IMPLANTS, AND TOOLS FOR FUSION OF SACROILIAC JOINTS

(71) Applicant: Sharad Rajpal, Boulder, CO (US)

(72) Inventor: Sharad Rajpal, Boulder, CO (US)

(21) Appl. No.: 15/959,567

(22) Filed: Apr. 23, 2018

Related U.S. Application Data
(60) Provisional application No. 62/488,335, filed on Apr. 21, 2017.

(51) Int. Cl. A61B 17/70; A61F 2/30

(52) U.S. Cl. CPC A61B 17/7055; A61F 2/30988; A61F 2310/00023; A61F 2002/30622; A61F 2002/30995

(57) ABSTRACT

A method of fusing a sacroiliac joint includes inserting expandable implants into multiple implant-receiving holes in ilium and sacrum; expanding the implants to engage both bones; and permitting bone growth to fuse the bones. Alternatively, implants may be inserted into multiple non-round implant-receiving holes in ilium and sacrum formed using a power impact driver attached to a broach tool to prevent movement of the sacroiliac joint. The expandable implants have an outer shell with bone-engagement ridges in porous outer surface to permit ingrowth of bone; an inner expander screw with forcing cone and threads; the outer shell portion having threads engaged with threads of the inner expander screw and an inner conical mating surface for the forcing cone of the inner expander screw; the outer shell engaging an outer sleeve of an insertion tool; and the inner expansion screw engaging a rotatable bit of an insertion tool.

### PRIORITY CLAIM

The present document claims priority to U.S. Provisional Patent Application No. 62/488,335, filed Apr. 21, 2017, the contents of which are hereby incorporated by reference.

### BACKGROUND

The sacroiliac joint, between the sacrum and the ilium bones of the pelvic girdle, is illustrated in FIGS. 1 and 2. Problems with this joint are involved in a significant percentage of people suffering from low back and/or sacroiliac joint pain.

A surgical relief procedure for sacroiliac pain involves stabilizing the sacroiliac joint by placing several implants, such as are illustrated in FIG. 3, across the sacroiliac joint as illustrated in FIG. 4. The implants prevent relative motion between sacrum and ilium at the sacroiliac joint. As the joint surfaces are disrupted and bone fragments enter the joint space during insertion of the implants, the sacrum and ilium fuse together after insertion of the implants as bone grows across the joint.

A prior procedure for insertion of the iFuse (trademark of SI-Bone, San Jose, Calif.) implant system is illustrated in FIG. 5. In this procedure, a surgeon opens a passage to a patient's ilium, inserts a tubular shield, and places a guide pin at a first location. The surgeon then drills a pilot hole over the guide pin through the ilium and into a first location in the sacroiliac joint into the sacrum using an electric drill. Since the pilot hole is round in cross-section, and the iFuse implant is triangular in cross section, and a tight fit of implant to hole is desired, the surgeon then inserts a triangular cutting tool, or broach, illustrated in FIG. 3A, and hammers this cutting tool through the ilium, sacroiliac joint space, and into the sacrum. The surgeon then pulls the cutting tool out of the resulting triangular hole in ilium and sacrum, and hammers an implant into the hole so the implant bridges the sacroiliac joint. The guide pin is then removed as is the tubular shield.

A hand-held hammer is typically used to hammer the cutting tool through the bone. Considerable force is required during the steps of hammering the cutting tool through the ilium, joint, and sacrum, the step of pulling the cutting tool out of the resulting hole, and the step of hammering the implant into the hole.

As a single implant is insufficient to stabilize the sacroiliac joint, the surgeon then removes the tubular shield and repeats the process to insert at least a second implant at a second location in the sacroiliac joint.

As shown in FIG. 3, the implants used in the iFuse system have a rough titanium surface that promotes the ingrowth of bone to ensure that, after a healing time, there is firm adhesion of the implant to both the sacrum and ilium as well as across the sacroiliac joint itself.

In an alternative sacroiliac joint stabilization system, several threaded screws, as illustrated in FIG. 6, are placed across the sacroiliac joint. While drilling a hole in sacrum and ilium is still necessary with this system, the holes remain round in cross section and there is no need for the broach of the iFuse system.

### SUMMARY

In an embodiment, method of fusing a sacroiliac joint includes forming at least two implant-receiving holes in ilium and sacrum; inserting an expandable implant into each implant-receiving hole; expanding the implant to engage the implant into both ilium and sacrum; and permitting bone growth to fuse the sacrum and ilium.

In another embodiment, a method of fusing a sacroiliac joint includes forming a non-round implant-receiving hole in ilium and sacrum using a power impact driver attached to a broach tool; inserting an implant into the implant-receiving hole; and repeating the steps of forming an implant-receiving hole, and inserting an implant in at least one other location on ilium, sacroiliac joint, and sacrum to prevent rotation of the sacroiliac joint.

In another embodiment, an expandable implant for fusing a joint has an outer shell with ridges in a porous outer surface adapted to permit ingrowth of bone and adapted to engage a first and a second bone; an inner expander screw with a forcing cone and a threaded portion; the outer shell portion threads engaged with the inner expander screw and an inner conical mating surface for the forcing cone of the inner expander screw; the outer shell adapted to engage an outer sleeve of an insertion tool; and the inner expansion screw adapted to engage a rotatable bit of an insertion tool.

### BRIEF DESCRIPTION OF THE FIGURES

FIG. 1 (source Wikipedia) is a posterior view of a pelvis showing a sacroiliac joint.

FIG. 2 (source Wikipedia) is an overhead view of pelvis showing a sacroiliac joint.

FIG. 3 illustrates implants of the PRIOR ART sacroiliac fusion system known as iFuse produced by SI-Bone.

FIG. 3A is a photograph of a PRIOR ART triangular hole cutter, or broach, used to cut holes in the ilium and sacrum when inserting the implant of FIG. 3.

FIG. 4 illustrates PRIOR ART iFuse implants in place across the sacroiliac joint.

FIG. 5 illustrates a PRIOR ART method of implanting the iFuse implant system.

FIG. 6 illustrates a PRIOR ART screw-based stabilization system for the sacroiliac joint.

FIG. 7 illustrates an impact tool coupled to a broach used for preparing a triangular hole in sacrum and ilium.

FIG. 8 illustrates a radially-expandable implant for stabilization of the sacroiliac joint.

FIG. 9 illustrates a shield portion of a radially-expandable implant having a springy portion linking multiple segments together.

FIG. 10 is an end view illustrating a shield portion of a radially-expandable implant having square cross section.

FIG. 11 is an end view illustrating a shield portion of a radially-expandable implant having triangular cross section.

FIG. 12 is a flowchart of the method of fusing a sacroiliac joint.

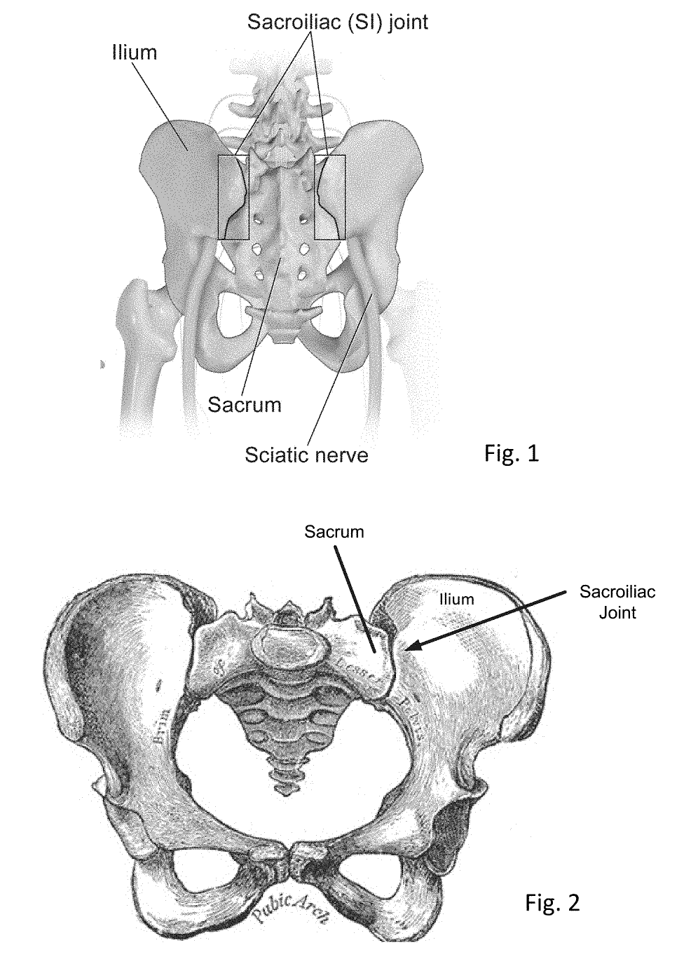

### DETAILED DESCRIPTION OF THE EMBODIMENTS

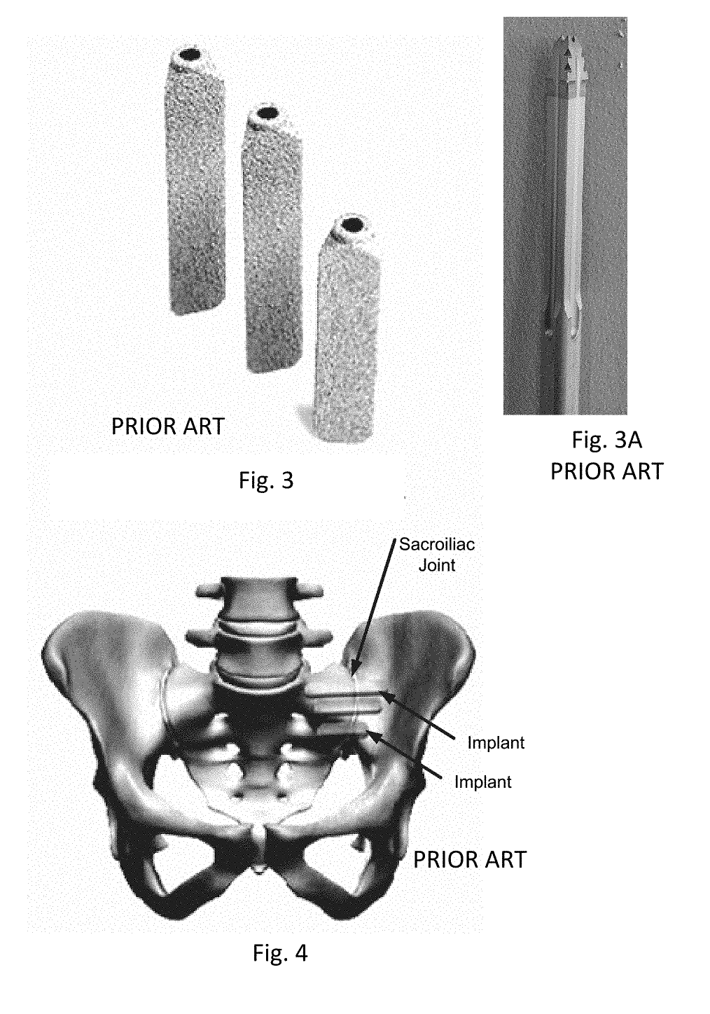

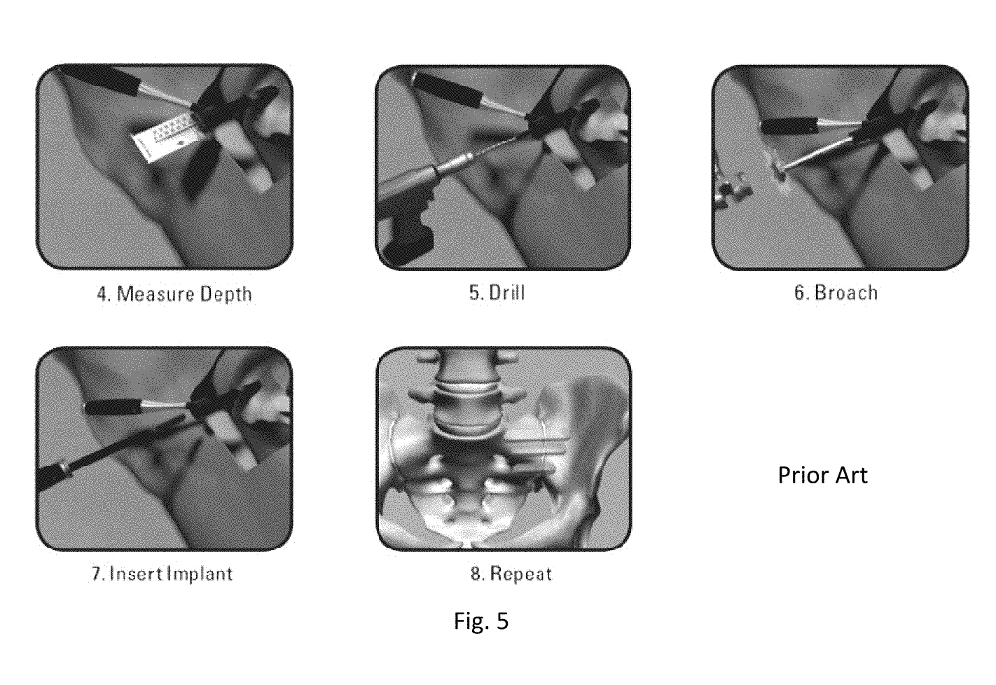

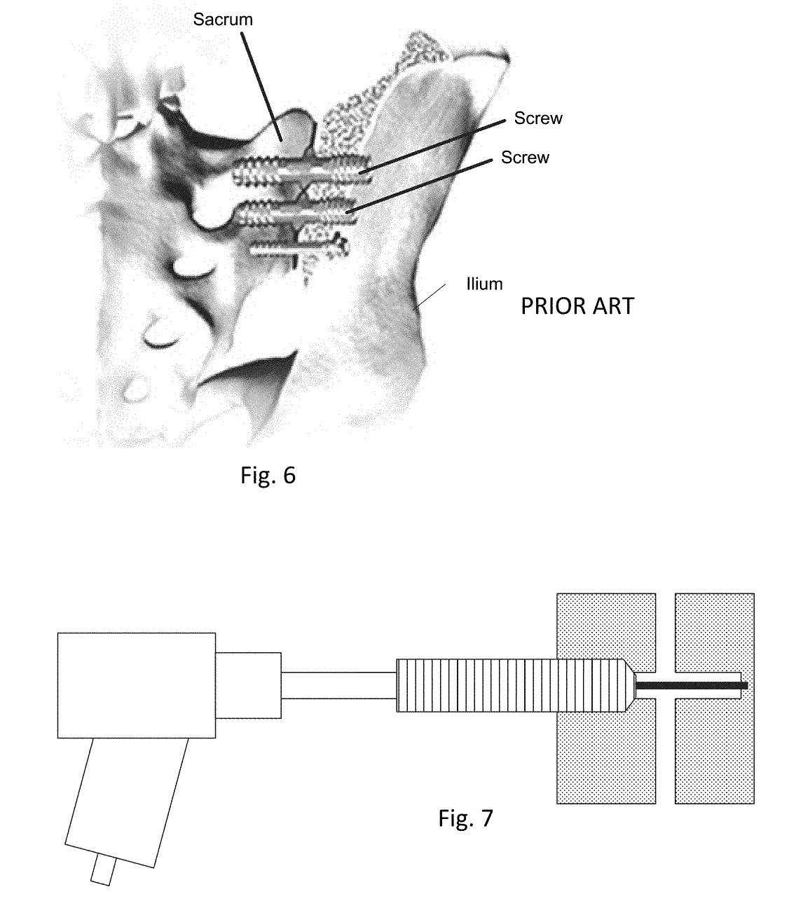

In an embodiment, instead of using a hand-held hammer to widen the pilot hole into the triangular hole, a power impact driver tool is used to drive the broach as illustrated in FIG. 7. The power tool is an electric or compressed air powered device providing successive impacts, in a manner similar to those provided by a small compressed-air chisel, miniaturized miner's drill, small electric chisel, or miniature electric jackhammer. For purposes of this document, the term impact driver includes compressed-air or electric powered devices that provide a series of small impacts when activated. In a particular embodiment, the power tool is used with the prior nonexpendable implant of FIG. 3.

With this embodiment, a method of fusing the sacroiliac joint includes making an incision and inserting a tubular cannula extending from skin to an exposed spot on the ilium where the fusion device will be inserted. A first pilot hole is drilled into the ilium and sacrum, and a guide wire is inserted. Next, a larger pilot hole centered on the guide wire is drilled in ilium and sacrum using an electric drill inserted through the cannula. Then an impact driver attached to a broach tool is inserted through the cannula and used to enlarge the pilot hole to form an implant-receiving hole in ilium and sacrum. After removing the impact driver and broach, an implant is inserted into the implant-receiving hole using the impact driver. The cannula is then removed and the method is repeated in at least one other location to place another implant extending from ilium through sacroiliac joint into sacrum to prevent rotation of the sacroiliac joint around an axis of the implant.

In an alternative embodiment, where the broach tool serves as a punch, the drilling of the pilot hole is omitted and the broach tool is driven through the bones to create the implant-receiving hole.

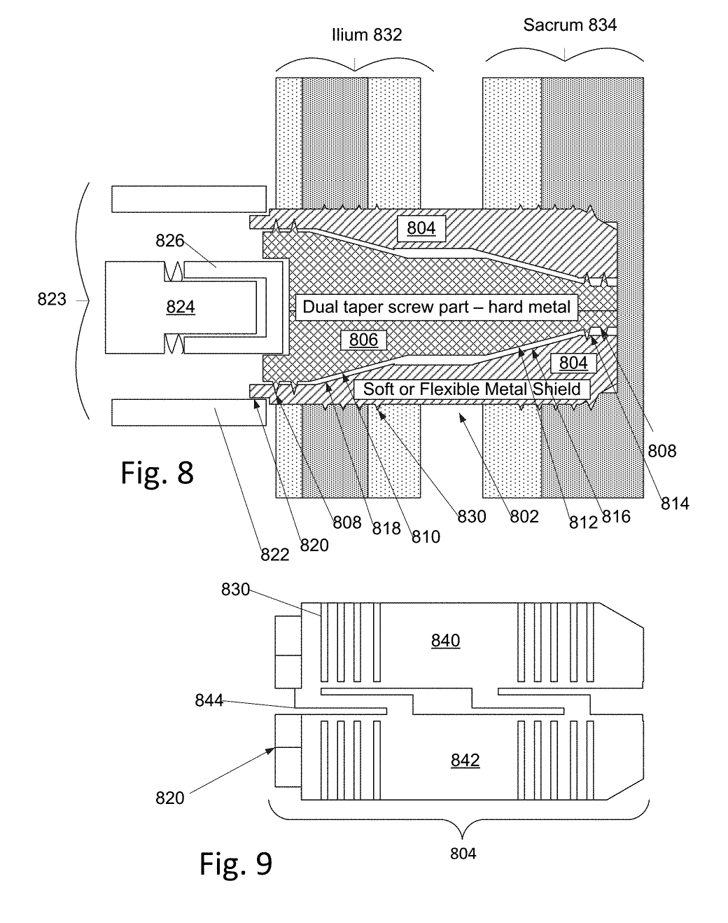

In another embodiment, an expandable implant 802 is used to stabilize the sacroiliac joint (FIG. 8) after holes are drilled. The expandable implant has an outer, shield, portion 804 fabricated of a soft or springy metal, such as a titanium alloy. The shield portion 804 of the implant is formed of a biocompatible alloy, or alternatively coated with a biocompatible alloy. In an alternative embodiment, the shield portion is formed of a biocompatible plastic.

The expandable implant has an inner expander-screw 806 formed of a hard, biocompatible, metal such as cobalt-chromium alloy, certain hard titanium alloys or of a hard, strong, biocompatible ceramic such as zirconia. The inner expander-screw 806 has at least one threaded portion 808 that engages the shield portion 804. The inner expander-screw 806 is formed with sacral 810 and ilial 812 forcing cones. The outer shield portion 804 has at least one threaded portion 814 that engages screw 806 threaded portion 808, and conical portions 816, 818 are configured to engage forcing expander-screw 806 forcing cones 810, 812. The shield portion has an engagement portion 820, that in a particular embodiment is polygonal, configured to engage a stationary portion 822 of an insertion tool 823. In an alternative embodiment, shield portion 822 has radial cuts (not shown) that engage mating radial ridges on an end of stationary portion 822 of insertion tool 823, the radial ridges and cuts adapted to securely prevent rotation of shield portion 822 despite radial movement of segments of shield portion 822. The insertion tool 823 also has a rotating portion 824 with a spring-loaded bit 826. Insertion tool 823 may in various embodiments be manually or electrically operated.

The shield portion 804 of the implant, as seen in a side view (FIG. 9), has multiple ridges 830 configured to, upon expansion of the implant, lock the implant into holes in the ilium 832 sacrum 834, or preferably both ilium 832 and sacrum 834. In embodiments where shield portion 804 is fabricated from soft-metal or plastic, shield portion 804 may be fabricated in one piece, in embodiments where shield portion 804 is fabricated of springy metal, shield portion 804 has multiple segments 840, 842 joined by slender bridges 844, bridges 844 being sufficiently flexible to permit separation of segments 840, 842 as expander-screw 806 is tightened into shield portion 804. In a particular embodiment, bridges 844 serve as springs that, upon withdrawal of expander-screw 806, retract segments 840, 842 and permit withdrawal of the expandable implant from the bone.

The expandable implant may be round in cross section.

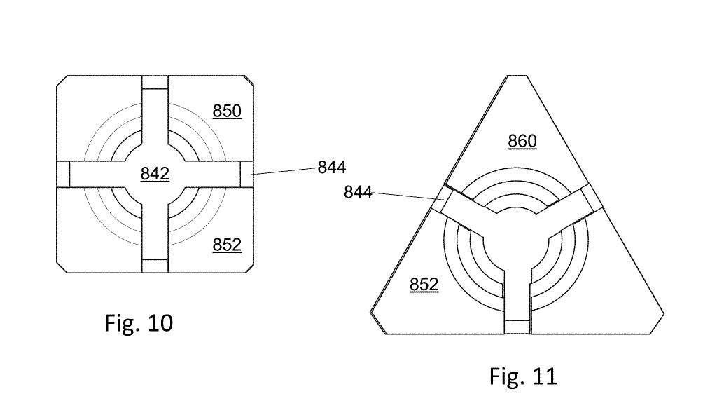

As illustrated in FIG. 10, in an alternative embodiment, the expandable implant shield portion is square in cross section with corners chamfered. As illustrated in FIG. 11, in another alternative embodiment the expandable implant shield portion is triangular in cross section with corners chamfered. In yet another alternative embodiment, the expandable implant shield has another shape such as a hexagonal cross section.

Insertion of the round expandable implant requires first exposing the ilium, then drilling holes through ilium 832 and into sacrum 834 of diameter such that implant 802 will fit into the holes. The implant is then inserted into the holes with the insertion tool stationary portion attached to the shield portion 804, and central rotating portion attached to the expander-screw portion. The expander-screw portion 806 is then tightened into shield portion 806, expanding the shield portion into firm contact with both the ilium and sacrum. To prevent rotation about an axis of the implant as could occur if a single implant were used, two or more implants are used for each sacroiliac joint, in a particular embodiment three implants are used for each sacroiliac joint being stabilized.

In embodiments using square (FIG. 10) or triangular (FIG. 11) implant cross sections, a pilot hole is drilled through ilium 832 into sacrum 834. A square or triangular broach, similar to that of FIG. 3A, but driven by an impact tool as illustrated in FIG. 7, is then used to widen the pilot hole into an implant engagement hole in both ilium and sacrum; the broach is then removed from the patient. Once the broach is removed, the expandable implant of FIG. 8, attached to the insertion tool, is inserted into the holes in ilium and sacrum, bridging the iliosacral joint space. The shield portion 804 is prevented from rotating while the expander-screw portion 806 is tightened into the shield portion 804, thereby forcing segments 840, 842, 850, 852, 860, 862 (FIGS. 9, 10, 11) into intimate contact with bone. In particular embodiments, the shield portion 804 of the expandable implant has a porous titanium surface, and optionally holes in its surface leading to cavities within the implant, so that, as the bone of sacrum and ilium heals, the bone may grow into and osseointegrate with the implant. In particular embodiments, the expander-screw portion 806 of the implant also has a porous titanium surface so that, as the bone heals, the bone may grow into and osseointegrate with both portions of the implant.

In particular embodiments, the shield portion of the implant has a hydroxyapatite (HA) coating to encourage bonding with bone as the bone heals following implantation.

The expandable implant of FIGS. 8-11 has advantage over the nonexpendable implants of FIG. 3 because the implant of FIGS. 8-11 may be slightly smaller than a nonexpendable implant yet grip the bone with similar or greater firmness, while being easier to remove than the implant of FIG. 3.

If removal of the implant is necessary before osseointegration occurs, implant removal is performed by exposing the implant, attaching the insertion tool 823 to the implant with its outer stationary portion 822 engaging the outer shield 804 of the implant to prevent rotation of the outer shield 804 while engaging the expander screw 806 with a rotatable spring-loaded bit 826 of the insertion tool 823, and rotating the spring-loaded bit to unscrew the expander screw 806, this allows the springy bridges 844 to withdraw segments 840, 842 from contact with the bone. A retriever tool having a handle, (not shown) is then screwed into a threaded portion 808 of the shield portion of the implant 804 and the shield portion is then wiggled free of scrum and ilium.

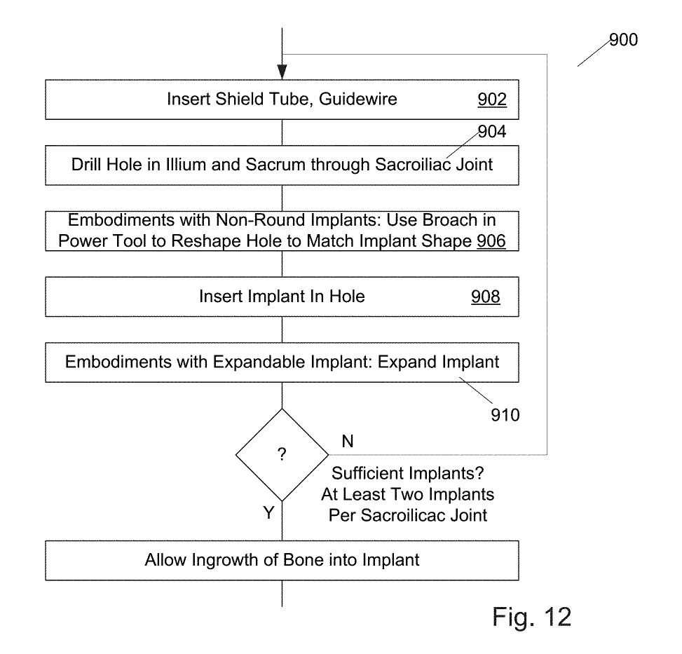

The method 900 (FIG. 12) of fusing the sacroiliac joint includes insertion of the shield tube 902 and guide wire, followed by drilling 904 a hole through from illium through sacroiliac joint into sacrum. In embodiments using a non-round implant, such as the implants of FIG. 10, or 11, with triangular or square cross-section, a broach having similar non-round shape to the implant is used with a power impact tool as illustrated in FIG. 7 to reshape 906 the hole to match and accept the implant. The implant is then attached to the insertion tool and inserted 908 into the hole bridging the sacroiliac joint and engaging with the hole in both ilium and sacrum. In embodiments using the expandable implants herein described, the implant is then expanded 910. If 912 sufficient implants have been placed, generally at least two and in some embodiments three implants per sacroiliac joint, the procedure ends, otherwise the procedure of implant insertion is repeated beginning with inserting a shield and guidewire 902 and drilling a hole 904 for another implant.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method of fusing a sacroiliac joint comprising:
forming an implant-receiving hole in ilium and sacrum;
inserting an expandable implant into the implant-receiving hole in ilium and sacrum;
expanding the expandable implant to engage the expandable implant into both ilium and sacrum;
repeating the steps of forming an implant-receiving hole, inserting an expandable implant, and expanding the expandable implant in at least one other location on ilium and sacrum to prevent rotation of the sacroiliac joint; and
permitting bone growth to fuse the sacrum and ilium.

2. The method of claim 1 wherein the implant is a non-round implant, and further comprising using a power impact driver with a broach tool to form the implant-receiving hole to a same shape as the non-round implant.

3. The method of claim 2 wherein expanding the implant to form a tight fit in the implant-receiving hole comprises
attaching an outer sleeve of an expander tool to an outer shell portion of the expandable implant;
attaching a rotatable bit of the expander tool to an inner expander screw of the implant; and
driving the inner expander screw into the implant.

4. The method of claim 3 wherein the expandable implant expands to form a tight fit in both the sacrum and the ilium.

5. The method of claim 1 wherein expanding the implant to form a tight fit in the implant-receiving hole comprises
attaching an outer sleeve of an expander tool to an outer shell portion of the expandable implant;
attaching a rotatable bit of the expander tool to an inner expander screw of the implant; and
driving the inner expander screw into the implant.

6. The method of claim 5 wherein the expandable implant expands to form a tight fit in both the sacrum and the ilium.

7. The method of claim 1 wherein the expandable implant comprises:
an outer shell portion having a plurality of ridges adapted to engage the outer shell portion into ilium and sacrum;
an inner expander screw having at least one forcing cone and at least one threaded portion;
the outer shell portion having at least one threaded portion engaged with the at least one threaded portion of the inner expander screw and formed with an inner conical mating surface for the at least one forcing cone of the inner expander screw;
the outer shell portion having a first end adapted to engage with an outer sleeve of an insertion tool;
the outer shell portion formed of a soft or springy metal having a porous outer surface adapted to permit ingrowth of bone; and
the inner expansion screw has a first end adapted to engage with a rotatable bit of an insertion tool.

8. The method of claim 7 wherein expanding the implant to form a tight fit in the implant-receiving hole comprises
attaching the outer sleeve of the expander tool to the outer shell portion of the expandable implant;
attaching the rotatable bit of the expander tool to the inner expander screw of the implant; and
driving the inner expander screw into the implant.

9. A method of fusing a sacroiliac joint comprising:
forming a non-round implant-receiving hole in ilium and sacrum using a power impact driver attached to a broach tool;
inserting an implant into the implant-receiving hole; and
repeating the steps of forming a non-round implant-receiving hole, and inserting an implant in at least one other location on ilium, sacroiliac joint, and sacrum to prevent rotation of the sacroiliac joint.

10. The method of claim 9 wherein the implant is an expandable implant, and further comprising expanding the implant to form a tight fit in the implant-receiving hole.

11. The method of claim 10 wherein the expandable implant expands to form a tight fit in both the sacrum and the ilium.

12. The method of claim 10 wherein the implant comprises an outer shell portion comprising titanium with a porous outer surface configured to permit ingrowth of bone.

13. The method of claim 12 wherein the implant comprises an inner expansion screw having a forcing cone and a threaded portion, and the outer shell portion has an inner thread adapted to engage with the threaded portion of the inner expansion screw and a surface adapted to engage with the forcing cone of the inner expansion screw, and wherein expanding the implant comprises tightening the inner expansion into the inner thread of the outer shell portion.

14. The method of claim 9 wherein the step of inserting an implant into the implant receiving hole is performed using the power impact driver.

15. An expandable implant configured for fusing a joint comprising:
an outer shell portion having a plurality of ridges adapted to engage the outer shell portion into a first and a second bone;
an inner expander screw having at least one forcing cone and at least one threaded portion;
the outer shell portion having at least one threaded portion engaged with the at least one threaded portion of the inner expander screw and formed with an inner conical mating surface for the at least one forcing cone of the inner expander screw;
the outer shell portion having a first end adapted to engage with an outer sleeve of an insertion tool;
the outer shell portion formed of a soft or springy metal having a porous outer surface adapted to permit ingrowth of bone; and
the inner expansion screw having a first end adapted to engage with a rotatable bit of an insertion tool.

16. The expandable implant of claim 15 wherein the outer shell portion comprises titanium.

17. The expandable implant of claim 15 wherein the outer shell portion comprises a plurality of segments linked by springy bridges.

18. The expandable implant of claim 15, further comprising the insertion tool, the outer sleeve of the insertion tool engaged with the outer shell portion of the implant and the bit of the expander tool engaged with the inner expansion screw of the implant.
